(19) United States
(12) Patent Application Publication
(10) Pub. No.: US 2022/0218320 A1
(43) Pub. Date: Jul. 14, 2022

(54) INERTIAL IMPACT NEEDLE SAMPLING APPARATUSES, SYSTEMS, AND METHODS

(71) Applicant: Olympus Medical Systems Corporation, Hachioji-shi (JP)

(72) Inventor: David A. Herrin, Seattle, WA (US)

(21) Appl. No.: 17/574,148

(22) Filed: Jan. 12, 2022

Related U.S. Application Data
(60) Provisional application No. 63/136,228, filed on Jan. 12, 2021.

(51) Int. Cl. A61B 10/04; A61B 10/02

(52) U.S. Cl. CPC A61B 10/04; A61B 10/0283; A61B 2010/0225; A61B 2010/045; A61B 2010/0208; A61B 10/0233

(57) ABSTRACT

Various disclosed embodiments include illustrative apparatuses, systems, and methods for performing tissue sampling. In an illustrative embodiment, an illustrative apparatus includes a flexible sheath having a lumen, a needle slidably received within the lumen, and a force translator configured to translate a proximal biased force into a distal force of the needle at a distal end of the flexible sheath.

### BACKGROUND

The statements in this section merely provide background information related to the present disclosure and may not constitute prior art.

Needle aspiration devices, such as transbronchial needle aspiration (TBNA) devices, are used to collect samples from target tissue, such as lymph nodes, tumors and nodules, for analysis. When actuating the TBNA device, the applied actuating force applied at the needle may travel a tortuous path before arriving at the distal needle tip.

### BRIEF SUMMARY

Various disclosed embodiments include illustrative apparatuses, systems, and methods for performing tissue sampling.

In an illustrative embodiment, an apparatus includes a flexible sheath having a lumen, a needle slidably received within the lumen, and a force translator configured to translate a proximal biased force into a distal force of the needle at a distal end of the flexible sheath.

In another illustrative embodiment, a system includes an endoscope with a working channel and an apparatus receivable within the working channel. The apparatus includes a flexible sheath having a lumen, a needle slidably received within the lumen, and a force translator. The force translator is configured to translate a proximal biased force into a distal force of the needle at a distal end of the flexible sheath.

In another illustrative embodiment, a method includes inserting a needle device such that a distal tip of the needle device is adjacent target tissue, applying a proximal force to a force transmitter, applying a loading force to a biasing device based on the applied proximal force to the force transmitter, releasing the proximal force applied to the force transmitter, and transferring a distal force from the biasing device to the force transmitter. Based on the transferred distal force to the force transmitter, the method includes causing an impact force between the force transmitter and a force receiver. Based on the impact force, the method includes expelling a distal tip of a needle component attached to the force receiver outside of a needle device sheath and into the adjacent target tissue.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

Illustrative embodiments are illustrated in referenced figures of the drawings. It is intended that the embodiments and figures disclosed herein are to be considered illustrative rather than restrictive.

FIG. 1-1 is a side plan view of an aspiration device in a deactivated state.

FIG. 1-2 a side plan view of the aspiration device of FIG. 1-1 in an activated state.

FIG. 2 is a partial cross-sectional view of the aspiration device of FIG. 1-1.

FIG. 3 is a partial cross-sectional and partial hidden-line view of the device of FIG. 2.

FIG. 4 is an isometric view of a portion of the device of FIG. 2.

FIG. 5 is a partial hidden-line side view of a portion of the device of FIG. 2 in a first operational state.

FIG. 6 is a partial hidden-line side view of a portion of the device of FIG. 2 in a second operational state.

FIG. 7 is a partial hidden-line side view of a portion of the device of FIG. 2 in a third operational state.

FIG. 8 is a flow chart of an illustrative method performed by the aspiration device of FIG. 1-1.

Like reference symbols in the various drawings generally indicate like elements.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Various disclosed embodiments include illustrative apparatuses, systems, and methods for performing tissue sampling.

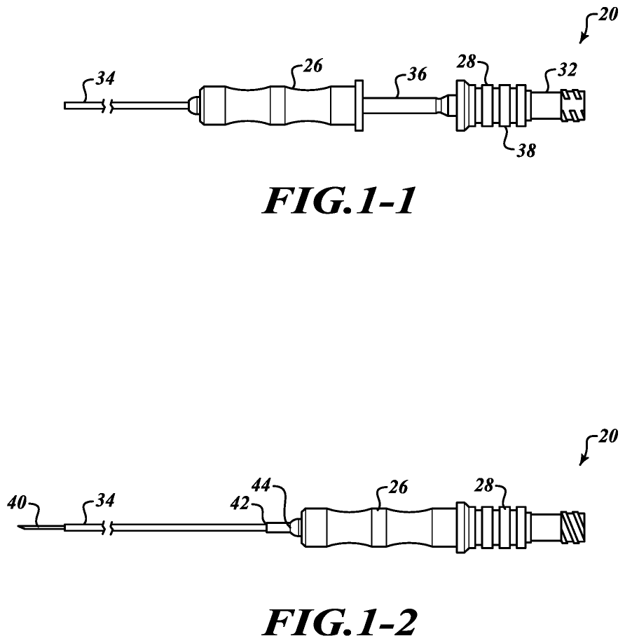

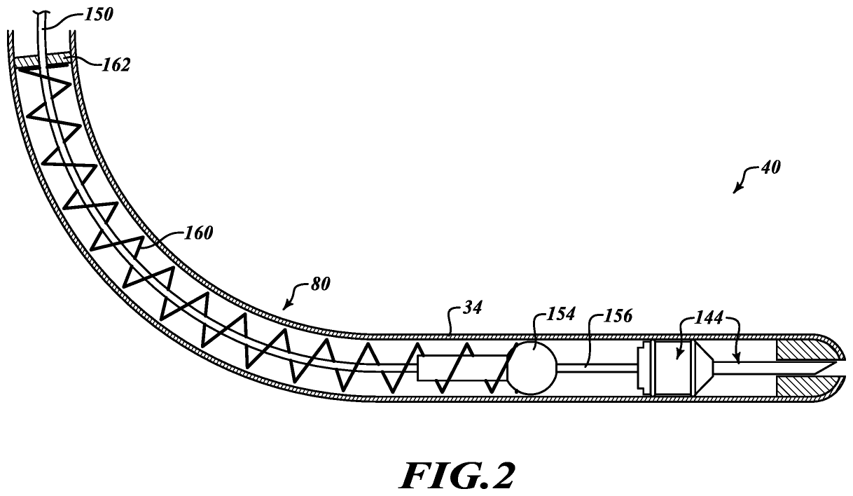

Referring to FIGS. 1-1 and 1-2, an illustrative needle aspiration device 20 (e.g., transbronchial needle aspiration (TBNA) device) is in a deactivated position (as shown in FIG. 1-1) and the device 20 is in an activated position (as shown in FIG. 1-2). The device 20 includes a handle body 26, a needle actuator 28, and a Luer component 32. The handle body 26 is attached to a proximal end of a sheath 34. The needle actuator 28 includes a shaft portion 36 coupled to a handle portion 38. The needle actuator 28 is attached to a pull wire (not shown) that is coupled to a needle drive assembly (not shown) within a distal end of the sheath 34. The Luer component 32 is attached to the needle actuator 28.

In the deactivated position (FIG. 1-1), the distal end of a needle assembly 40 is retracted within the sheath 34. In the activated position (FIG. 1-2), the distal end of the needle assembly 40 is exposed beyond the distal end of the sheath 34.

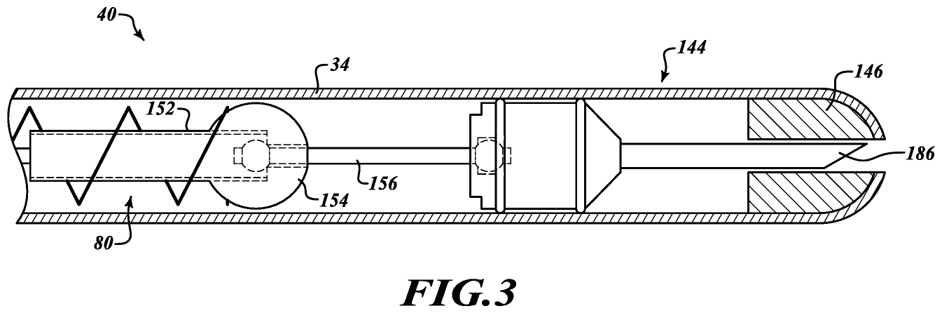

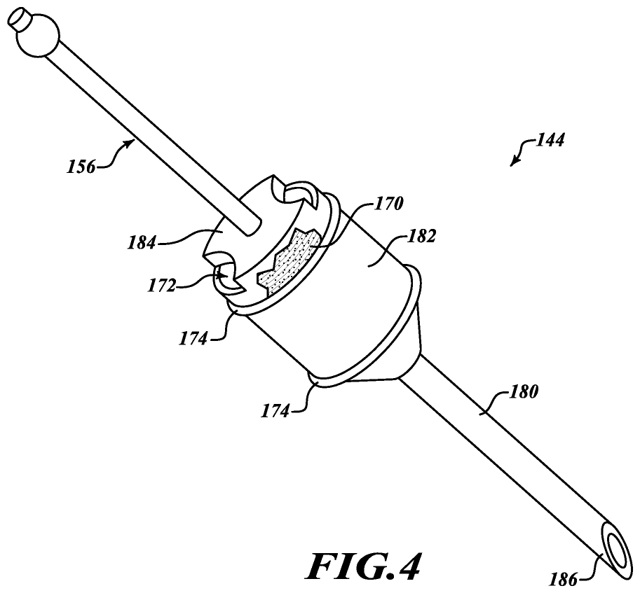

Referring additionally to FIGS. 2-4, in various embodiments a distal end of a needle assembly 40 is shown at a distal end of the sheath 34. The needle assembly 40 includes various components for applying a distal force to a needle tip. The needle assembly 40 includes a force transmitter device 80 having a pull wire 150, a biasing device 160, a stop collar 162, and a ball 154. The needle assembly 40 also includes a force receiver device 144 that is connected by a slack link line 156 to the force transmitter device 80. The force receiver device 144 includes a needle 180 having a sharpened distal tip 186, a biopsy housing 182, and a cap 184. The sheath 34 includes a removable distal tip 146 that includes a lumen that is sized to receive the needle 180. The needle 180 includes a lumen. A distal end of the biopsy housing 182 is connected to a proximal end of the needle 180. The biopsy housing 182 includes a reservoir that is in fluid communication with the lumen of the needle 180. The cap 184 is removably attachable to a proximal end of the biopsy housing 182.

In various embodiments, the cap 184 has one or more passageways 172. The cap 184 or the biopsy housing 182 includes a filter 170 for allowing tissue samples to remain within the biopsy housing 182, while allowing a proximal suction force to be applied to the biopsy housing 182. The passageway 172 allows for a suction force to pass from within the biopsy housing 182 to a proximal side of the cap 184. In various embodiments, the suction force may be created by a suction device, such as a syringe, attached to the Luer component 32. The biopsy housing 182 may include one or more seals 174 that are located on the outer circumference of the biopsy housing 182. Thus, the suction force is applied within the biopsy housing 182 due to the sealing of the area around the biopsy housing 182.

In various embodiments, the slack link line 156 includes knobs or beads attached at either end. The cap 184 includes a lumen for receiving one of the knobs and the slack link line 156. The cap 184 includes a second lumen proximately from the first lumen that has a cross-sectional dimension that is smaller than a cross-sectional dimension of the knob. The ball 154 includes a proximal lumen and a distal lumen. The proximal lumen is sized to receive the slack link line 156 and the knob. The distal lumen has a cross-sectional dimension that is smaller than a cross-sectional dimension of the knob.

In various embodiments, a hollow tube 152 attaches at a proximal end to a distal end of the pull wire 150 and is attached to or is received within the ball 154. The hollow tube 152 may allow the knob of the slack link line 156 to maintain its position within the sheath 34 as the ball 154 and the hollow tube 152 move distally.

In various embodiments, the biasing device 160 may be located between the stop collar 162 and the ball 154. The biasing device 160 may be a spring that is compressed by the ball 154 when an operator pulls the pull wire 150 proximally, thus compressing the spring against the stop collar 162.

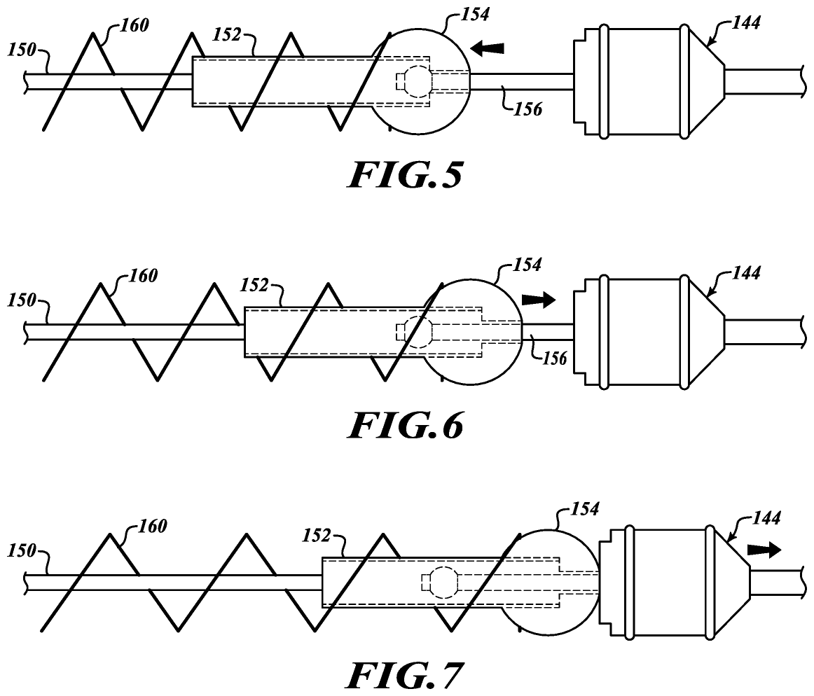

Referring additionally to FIG. 5, as the operator pulls the pull wire 150 proximately, the slack link line 156 becomes taut thus pulling the force receiver device 144 proximately. The sharpened distal tip 186 is thus retracted within the sheath 34.

Referring additionally to FIGS. 6 and 7, the operator has released the proximal force on the pull wire 150 as shown in FIG. 6. This causes the spring to force the ball 154 distally until, as shown in FIG. 7, the ball 154 makes contact with the force receiver device 144. The force of contact by the ball 154 on the force receiver device 144 causes the force receiver device 144 with the sharpened distal tip 186 to move distally and for the sharpened distal tip 186 to extend out of the end of the sheath 34 and into any surrounding tissue.

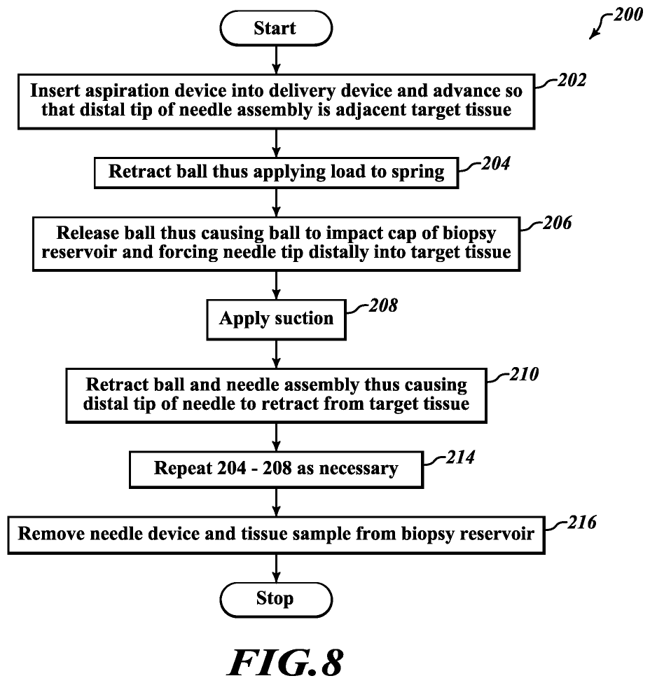

Referring additionally to FIG. 8, in various embodiments an illustrative method 200 is performed by the needle aspiration device 20. At a block 202 the device 20 is inserted into a delivery device in advance of the sharpened distal tip 186 and the distal end of the sheath 34 is adjacent to target tissue. At a block 204, an operator retracts the ball 154 by pulling the needle actuator 28 proximally relative to the handle body 26, thus applying a load to the biasing device 160. At a block 206, the operator releases the proximal force on the ball 154, thus causing the ball 154 to be forced distally by the biasing device 160, thus making contact with the biopsy housing 182. The contact force onto the biopsy housing 182 causes the biopsy housing 182 to also move distally, whereby the sharpened distal tip 186 extends out of the distal tip of the sheath 34 and into surrounding target tissue. At a block 208, the operator applies a suction force using a suction device attached to the needle actuator 28. At a block 210, the operator retracts the ball 154 and the biopsy housing 182 by retracting the needle actuator 28 proximately relative to the handle body 26. This results in the distal tip of the sharpened distal tip 186 to be retracted into the sheath 34. At a block 214, the steps at blocks 204-208 are repeated as desired. At a block 216, after the operator has determined that tissue sampling is complete, the needle aspiration device 20 is removed from the patient and any tissue samples received within the biopsy housing 182 are removed for analysis.

It will be appreciated that the ball 154 may be composed of multiple balls or may be non-spherical devices.

From the foregoing discussion and associated drawing figures, it will be appreciated that various embodiments have been disclosed and illustrated. To that end and without any implication of any limitation (which is not to be inferred), the following paragraphs set forth non-limiting summaries of various embodiments disclosed herein by way of example only and not of limitation:

A. An apparatus comprising: a flexible sheath including a lumen; a needle slidably received within the lumen; and a force translator configured to translate a proximal biased force into a distal force of the needle at a distal end of the flexible sheath.

B. The apparatus of A, wherein: the force translator includes: a stop configured to be attached to a wall of the lumen; a pull wire configured to be received within the lumen, the a force transmitter configured to be attached to a distal end of the pull wire; a biasing device disposed between the stop and the force transmitter; and the needle includes: a piercing tip having a lumen; and a force receiver housing attachable to a proximal end of the piercing tip, the force receiver having a distal end configured to be attached to a proximal end of the piercing tip.

C. The apparatus of B, further comprising: a needle handle including: a handle body couplable to a proximal end of the flexible sheath; and an actuator portion couplable to a proximal end of the pull wire.

D. The apparatus of B, wherein: the force transmitter further includes: a first lumen; and a second lumen in fluid communication with the first lumen, the second lumen being distal of the first lumen and having a smaller cross-sectional dimension than the first lumen; the force receiver housing further includes: a third lumen; and a fourth lumen in fluid communication with the third lumen, the fourth lumen being proximal of the third lumen and having a smaller cross-the sectional dimension than the third lumen; and the apparatus further comprises a slack wire including: a flexible wire; a first knob attachable to a proximal end of the flexible wire; and a second knob attachable to a distal end of the flexible wire, the first and second knobs having a dimension that is greater than the cross-sectional dimension of the second lumen and the fourth lumen.

E. The apparatus of D, wherein the force transmitter includes a steel ball.

F. The apparatus of E, wherein the force transmitter further includes a tube configured to extend proximally from the ball, wherein the lumen of the force transmitter is contiguous between the ball and the tube, the tube having a proximal end couplable to a distal end of the pull wire.

G. The apparatus of G, wherein the force receiver housing further includes: a cap including a proximal face configured to make contact with the ball; and a sample chamber attachable to the cap and configured to receive samples from the lumen of the piercing tip.

H. The apparatus of G, wherein: the cap further includes a longitudinal air passageway; and the force receiver housing further includes a filter configured to allow air transmission through the longitudinal air passageway of the cap.

I. The apparatus of G, wherein the force receiver housing further includes one or more seals configured to provide a seal between a distal side of the force receiver housing and a proximal side of the force receiver housing within the flexible sheath.

J. The apparatus of G, wherein the proximal face is shaped to be conformable with a shape of the ball.

K. A system comprising: an endoscope including a working channel; and an apparatus receivable within the working channel, the apparatus including: a flexible sheath including a lumen; a needle slidably received within the lumen; and a force translator configured to translate a proximal biased force into a distal force of the needle at a distal end of the flexible sheath.

L. The system of K, wherein: the force translator includes: a stop configured to be attached to a wall of the lumen; a pull wire configured to be received within the lumen; a force transmitter configured to be attached to a distal end of the pull wire; a biasing device disposed between the stop and the force transmitter; and the needle includes: a piercing tip having a lumen; and a force receiver housing attachable to a proximal end of the piercing tip, the force receiver having a distal end configured to be attached to a proximal end of the piercing tip.

M. The apparatus of L, further comprising: a needle handle including: a handle body couplable to a proximal end of the flexible sheath; and an actuator portion couplable to a proximal end of the pull wire, wherein: the force transmitter further includes: a first lumen; and a second lumen in fluid communication with the first lumen, the second lumen being distal of the first lumen and having a smaller cross-sectional dimension than the first lumen; the force receiver housing further includes: a third lumen; and a fourth lumen in fluid communication with the third lumen, the fourth lumen being proximal of the third lumen and having a smaller cross-the sectional dimension than the third lumen; and the apparatus further comprises a slack wire including: a flexible wire; a first knob attachable to a proximal end of the flexible wire; and a second knob attachable to a distal end of the flexible wire, the first and second knobs having a dimension that is greater than the cross-sectional dimension of the second lumen and the fourth lumen.

N. The apparatus of M, wherein: the force transmitter includes a steel ball; and the force transmitter further includes a tube configured to extend proximally from the ball, the lumen of the force transmitter is contiguous between the ball and the tube, the tube having a proximal end couplable to a distal end of the pull wire.

O. The apparatus of N, wherein the force receiver housing further includes: a cap including a proximal face configured to make contact with the ball; and a sample chamber attachable to the cap and configured to receive samples from the lumen of the piercing tip.

P. The apparatus of O, wherein the force receiver housing further includes one or more seals configured to provide a seal between a distal side of the force receiver housing and a proximal side of the force receiver housing within the flexible sheath.

Q. A method comprising: inserting a needle device such that a distal tip of the needle device is adjacent target tissue; applying a proximal force to a force transmitter; applying a loading force to a biasing device based on the applied proximal force to the force transmitter; releasing the proximal force applied to the force transmitter; transferring a distal force from the biasing device to the force transmitter; based on the transferred distal force to the force transmitter, causing an impact force between the force transmitter and a force receiver; and based on the impact force, expelling a distal tip of a needle component attached to the force receiver outside of a needle device sheath and into the adjacent target tissue.

R. The method of Q, further comprising: retracting the force transmitter in a proximal direction; and retracting the force receiver in the proximal direction based on a slack line connection between the force transmitter and the force receiver.

S. The method of R, further comprising: applying a suction force within the needle device sheath; and responsive to the applied suction force, applying a proximal suction force at the distal tip of the needle component.

T. The method of Q, further comprising: removing a distal end of the needle device sheath; expelling the force receiver and the needle component from the needle device sheath; removing a cap of the force receiver; and removing a sample tissue disposed within a chamber of the force receiver.

In some instances, one or more components may be referred to herein as “configured to,” “configured by,” “configurable to,” “operable/operative to,” “adapted/adaptable,” “able to,” “conformable/conformed to,” etc. Those skilled in the art will recognize that such terms (for example “configured to”) generally encompass active-state components and/or inactive-state components and/or standby-state components, unless context requires otherwise.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (for example, “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (for example, the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented in a sequence(s), it should be understood that the various operations may be performed in other orders than those which are illustrated or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

While the disclosed subject matter has been described in terms of illustrative embodiments, it will be understood by those skilled in the art that various modifications can be made thereto without departing from the scope of the claimed subject matter as set forth in the claims.

What is claimed is:

1. An apparatus comprising:
a flexible sheath including a lumen;
a needle slidably received within the lumen; and
a force translator configured to translate a proximal biased force into a distal force of the needle at a distal end of the flexible sheath.

2. The apparatus of claim 1, wherein:
the force translator includes:
a stop configured to be attached to a wall of the lumen;
a pull wire configured to be received within the lumen, the
a force transmitter configured to be attached to a distal end of the pull wire;
a biasing device disposed between the stop and the force transmitter; and
the needle includes:
a piercing tip having a lumen; and
a force receiver housing attachable to a proximal end of the piercing tip, the force receiver having a distal end configured to be attached to a proximal end of the piercing tip.

3. The apparatus of claim 2, further comprising:
a needle handle including:
a handle body couplable to a proximal end of the flexible sheath; and
an actuator portion couplable to a proximal end of the pull wire.

4. The apparatus of claim 2, wherein:
the force transmitter further includes:
a first lumen; and
a second lumen in fluid communication with the first lumen, the second lumen being distal of the first lumen and having a smaller cross-sectional dimension than the first lumen;
the force receiver housing further includes:
a third lumen; and
a fourth lumen in fluid communication with the third lumen, the fourth lumen being proximal of the third lumen and having a smaller cross-the sectional dimension than the third lumen; and
the apparatus further comprises a slack wire including:
a flexible wire;
a first knob attachable to a proximal end of the flexible wire; and
a second knob attachable to a distal end of the flexible wire, the first and second knobs having a dimension that is greater than the cross-sectional dimension of the second lumen and the fourth lumen.

5. The apparatus of claim 4, wherein the force transmitter includes a steel ball.

6. The apparatus of claim 5, wherein the force transmitter further includes a tube configured to extend proximally from the ball, wherein the lumen of the force transmitter is contiguous between the ball and the tube, the tube having a proximal end couplable to a distal end of the pull wire.

7. The apparatus of claim 6, wherein the force receiver housing further includes:
a cap including a proximal face configured to make contact with the ball; and
a sample chamber attachable to the cap and configured to receive samples from the lumen of the piercing tip.

8. The apparatus of claim 7, wherein:
the cap further includes a longitudinal air passageway; and
the force receiver housing further includes a filter configured to allow air transmission through the longitudinal air passageway of the cap.

9. The apparatus of claim 7, wherein the force receiver housing further includes one or more seals configured to provide a seal between a distal side of the force receiver housing and a proximal side of the force receiver housing within the flexible sheath.

10. The apparatus of claim 7, wherein the proximal face is shaped to be conformable with a shape of the ball.

11. A system comprising:
an endoscope including a working channel; and
an apparatus receivable within the working channel, the apparatus including:
a flexible sheath including a lumen;
a needle slidably received within the lumen; and
a force translator configured to translate a proximal biased force into a distal force of the needle at a distal end of the flexible sheath.

12. The system of claim 11, wherein:
the force translator includes:
a stop configured to be attached to a wall of the lumen;
a pull wire configured to be received within the lumen;
a force transmitter configured to be attached to a distal end of the pull wire;
a biasing device disposed between the stop and the force transmitter; and
the needle includes:
a piercing tip having a lumen; and
a force receiver housing attachable to a proximal end of the piercing tip, the force receiver having a distal end configured to be attached to a proximal end of the piercing tip.

13. The apparatus of claim 12, further comprising:
a needle handle including:
a handle body couplable to a proximal end of the flexible sheath; and
an actuator portion couplable to a proximal end of the pull wire,
wherein:
the force transmitter further includes:
a first lumen; and
a second lumen in fluid communication with the first lumen, the second lumen being distal of the first lumen and having a smaller cross-sectional dimension than the first lumen;
the force receiver housing further includes:
a third lumen; and
a fourth lumen in fluid communication with the third lumen, the fourth lumen being proximal of the third lumen and having a smaller cross-the sectional dimension than the third lumen; and
the apparatus further comprises a slack wire including:
a flexible wire;
a first knob attachable to a proximal end of the flexible wire; and
a second knob attachable to a distal end of the flexible wire, the first and second knobs having a dimension that is greater than the cross-sectional dimension of the second lumen and the fourth lumen.

14. The apparatus of claim 13, wherein:
the force transmitter includes a steel ball; and
the force transmitter further includes a tube configured to extend proximally from the ball, the lumen of the force transmitter is contiguous between the ball and the tube, the tube having a proximal end couplable to a distal end of the pull wire.

15. The apparatus of claim 14, wherein the force receiver housing further includes:
a cap including a proximal face configured to make contact with the ball; and
a sample chamber attachable to the cap and configured to receive samples from the lumen of the piercing tip.

16. The apparatus of claim 15, wherein the force receiver housing further includes one or more seals configured to provide a seal between a distal side of the force receiver housing and a proximal side of the force receiver housing within the flexible sheath.

17. A method comprising:
inserting a needle device such that a distal tip of the needle device is adjacent target tissue;
applying a proximal force to a force transmitter;
applying a loading force to a biasing device based on the applied proximal force to the force transmitter;
releasing the proximal force applied to the force transmitter;
transferring a distal force from the biasing device to the force transmitter;
based on the transferred distal force to the force transmitter, causing an impact force between the force transmitter and a force receiver; and
based on the impact force, expelling a distal tip of a needle component attached to the force receiver outside of a needle device sheath and into the adjacent target tissue.

18. The method of claim 17, further comprising:
retracting the force transmitter in a proximal direction; and
retracting the force receiver in the proximal direction based on a slack line connection between the force transmitter and the force receiver.

19. The method of claim 17, further comprising:
applying a suction force within the needle device sheath; and
responsive to the applied suction force, applying a proximal suction force at the distal tip of the needle component.

20. The method of claim 17, further comprising:
removing a distal end of the needle device sheath;
expelling the force receiver and the needle component from the needle device sheath;
removing a cap of the force receiver; and
removing a sample tissue disposed within a chamber of the force receiver.
